(19) United States
(12) Patent Application Publication
(10) Pub. No.: US 2008/0183146 A1
(43) Pub. Date: Jul. 31, 2008

(54) ABSORBENT ARTICLE

(76) Inventor: Martin MATUSHEK, West Dundee, IL (US)

Correspondence Address: LADAS & PARRY LLP, 224 SOUTH MICHIGAN AVENUE, SUITE 1600, CHICAGO, IL, 60604, US

(21) Appl. No.: 11/627,493

(22) Filed: Jan. 26, 2007

(51) Int. Cl. A61F 13/53

(52) U.S. Cl. 604/367

(57) ABSTRACT

An absorbent article for use in rapid and efficient absorption of various spilt liquid products is described. The absorbent article includes a relatively high specific heat capacity component so as to aid in minimizing any excessive temperature buildup brought about by the absorptive cleanup process. By minimizing exothermic temperature, undesirable exposure of any resultant foul smelling or noxious gases is also minimized during the absorptive process. The absorbent article includes a super-absorbent polymer and a heat-sink material.

### FIELD OF THE INVENTION

The present invention relates to absorbents, more particularly to an absorbent article, for use in cleanup operations of spilt liquid products while minimizing temperature increases brought about by the adsorptive process.

### DESCRIPTION OF THE PRIOR ART

A wide variety of absorbent articles is currently available on the commercial market. An even larger number of kits and products using these products are well known in the art of cleaning up and/or preventing liquid spills. For example, disposable diapers, adult incontinent pads and feminine hygiene pads provide well known examples of products that incorporate various absorbent articles that can absorb up to one hundred times their weight in excess moisture.

A number of approaches have been exploited to improve the capacity and efficiency of absorbent articles. For example, Roe disclosed in U.S. Pat. No. 5,419,956 that the addition of inorganic particles such as silica added to super-absorbent polymers could be used to improve fluid uptake rate and could increase fluid distribution. Sun disclosed in example U.S. Pat. No. 6,124,391 adding certain inorganic powders and even some waxes could admixed with super-absorbent polymers to enhance the absorptive properties of the absorbent.

However, there is still a need for an improved absorbent article having a high absorbent capacity which is also capable of simultaneously minimizing any noxious fumes that might occur when the spilled liquid is rapidly absorbed by the absorptive article. That is, the absorptive process is often an exothermic process that necessarily increases the temperature of the spilt liquid and increases the vapor pressure, i.e., the noxious odor, of the spilt liquid.

While all of the above-described devices fulfill their respective, particular objectives and requirements, the aforementioned patents do not describe an absorbent article composed of a super-absorbent polymer and a heat-sink material in which the heat-sink material has a relatively high specific heat capacity so as to aid in minimizing any excessive buildup of temperature brought about by the absorptive cleanup process.

This combination of elements would specifically match the user's particular individual needs of making it possible to provide a means for minimizing exothermic temperature buildup which in turn minimizes any undesirable exposure of any resultant foul smelling or noxious gases that are likely to be emitted during an absorptive exothermic process.

The above-described patents make no provision for an absorbent article composed of a super-absorbent polymer and a heat-sink material. Therefore, a need exists for a new and improved an absorbent article composed of a super-absorbent polymer and a heat-sink material. In this respect, the an absorbent article according to the present invention substantially departs from the conventional concepts and designs of the prior art, and in doing so provides an apparatus primarily developed for the purpose of providing a convenient means for making it possible to provide a means for minimizing temperature buildup brought about by the absorptive process in order to minimizes or avoid exposure to any noxious gases that can be emitted during the cleanup operation of the a spilt liquid product, such as a chlorine bleach spill.

### SUMMARY OF THE INVENTION

The present absorbent article of using, according to the principles of the present invention, overcomes a number of the shortcomings of the prior art by providing a novel absorbent article for use in absorbing spilt liquids while minimizing any excessive temperature buildup brought about by the absorptive cleanup process.

The absorbent article includes a super-absorbent polymer and a heat-sink material in which the heat-sink material is selected to have a relatively high specific heat capacity so as to aid in minimizing any excessive temperature buildup brought about by the absorptive cleanup process. By minimizing exothermic temperature any consequential undesirable exposure of a foul smelling or noxious gases is also minimized during the absorptive process.

In view of the foregoing disadvantages inherent in the known type absorbent articles now present in the prior art, the present invention provides an improved absorbent article, which will be described subsequently in great detail, is to provide a new and improved absorbent article which is not anticipated, rendered obvious, suggested, or even implied by the prior art, either alone or in any combination thereof.

To attain this, the present invention essentially comprises a super-absorbent polymer and a heat-sink material in which the heat-sink material has a relatively high specific heat capacity so as to aid in minimizing any excessive temperature buildup brought about by the absorptive cleanup process.

There has thus been outlined, rather broadly, the more important features of the invention in order that the detailed description thereof that follows may be better understood, and in order that the present contribution of the art may be better appreciated.

The invention may also include an optional vapor suppressant.

Numerous features and advantages of the present invention will be readily apparent to those of ordinary skill in the art upon reading of the following detailed description of presently preferred. In this respect, before explaining the current embodiment of the invention in detail, it is to be understood that the invention is not limited in its application to the details of construction and to the arrangements of the components set forth in the following description. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Therefore the present invention provides a new and improved absorbent article that has many of the advantages of the prior absorbent article and minimizing a number of their disadvantages.

The present invention also provides a new and improved absorbent article that may be easily and efficiently manufactured and marketed.

The present invention also provides a new and improved absorbent article that has a low cost of manufacture with regard to both materials and labor, and which accordingly is then susceptible to low prices of sale to the consuming public, thereby making the absorbent article economically available to the buying public.

The present invention also provides an absorbent article that has some of the advantages of the prior art, while simultaneously overcoming some of the disadvantages normally associated therewith.

Even still another aspect of the present invention is to provide absorbent article having a super-absorbent polymer and a heat-sink material in which the heat-sink material is selected to have a relatively high specific heat capacity so as to aid in minimizing any excessive temperature buildup brought about by the absorptive cleanup process.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying descriptive matter in which describe a number of preferred embodiments of the invention.

### DETAILED DESCRIPTION OF THE PREFERRED EMBODIMENT

One preferred embodiment of the absorbent article comprises a super-absorbent polymer and a heat-sink material. The super-absorbent polymer may be any commercially available super-absorbent polymer such as those selected from the group consisting of polyacrylate polymers, starch graft copolymers, cellulose graft copolymers, acrylate-acrylamide co-polymer, cross-linked carboxymethylcellulose derivatives, and admixtures thereof. One preferred super-absorbent polymer is that it comprises a lightly-crosslinked sodium polyacrylate having less than 15% by weight of extractables in the super-absorbent polymer. Still yet another preferred super-absorbent polymer is that it comprises a lightly-crosslinked sodium polyacrylate having less than 10% by weight of extractables in the super-absorbent polymer. The extractables comprise residues extracted from the super-absorbent polymer using an extracting solvent wherein the extracting solvent is a non polar solvent selected from the group consisting of a methylene chloride, toluene, benzene, cyclohexane, n-pentane, n-hexane and n-octane. The heat-sink material may be any commercially available heat-sink material as long as a reaction temperature of no more than 150° F. is generated when equal amounts of the absorbent article and a spilled liquid are mixed together.

One preferred embodiment of the absorbent article is that the heat-sink material contribute to restricting the generation of a reaction temperature to no more than 120° F. when approximately equal amounts of the absorbent article and a spilled liquid are mixed together. The particular heat-sink material of the absorbent article may be any material such as those selected from the group consisting of vermiculite, bentonite, hectorite, activated carbon, charcoal, mica, perlite and admixtures thereof as long as the specific heat of the heat-sink material is at least 0.5 kJ/kgK. One preferred embodiment of the heat-sink material is that it has a specific heat of at least 0.8 kJ/kgK.

The absorbent article is preferably designed so that it is capable of absorbing a substantial amount of a spilled liquid in which one preferred configuration is that the absorbent article has an overall absorbent capacity of over 60% by weight of the absorbent article. Still another preferred configuration is that the absorbent article has an overall absorbent capacity of over 150% by weight of the absorbent article.

The absorbent article is also preferably designed to minimize any unwanted emission of noxious gasses that may be emitted when the absorbent article absorbs the spilled liquid. The heat-sink material of the absorbent article is preferably designed to aid in efficiently controlling any excessive exothermic heat that might be generated during the adsorptive process so that the temperature of the composite mixture of the adsorptive article combined with the spilled liquid is consequently restricted to below 150 F whereby lowing the vapor pressure of any unwanted noxious gas that may be emitted. Another preferable configuration is that heat-sink material of the adsorptive article has a sufficiently high heat capacity>0.8 kJ/kgK and sufficiently high mass so that it limits the temperature of combining the adsorptive article combined with the spilled liquid to below 120° F.

The relative compositional makeup of the absorbent article may be any known ratio as long as the super-absorbent polymer is present in an amount of about 20% to about 80% by weight of the absorbent article and as long as the heat-sink material is present in an amount of about 20 to about 80% by weight of the absorbent article.

The geometric compositional makeup of the super-absorbent polymer may be any known size shape and phase such as a particulate powder or even a slurry. One preferred geometric makeup of the super-absorbent polymer is that it comprises a majority of particles sized to be greater than 700 μm in diameter.

The spilled liquid that the absorbent article is intended to absorb may be any spilled liquids such as those selected from the group consisting of a chlorine bleach product, an ammonia-based cleaning product, a pesticide product, a herbicide product, a fertilizer product, a motor oil product, a shampoo product and a milk product.

An optional vapor suppressant may be added to the absorbent article in which the optional vapor suppressant is selected from the group consisting of cellulosic fibers, sawdust, corncob particles, soybean husks, peat moss and admixtures thereof.

### EXAMPLE 1

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 100% dried corn cob particles | Rapid Cl₂ odor suppression. |
| (200–800 μm range) | 155° F. max temp observed. |
| | No residual sheen or slickness detected |
| | on floor after clean-up. |
| | No difficulty in sweeping up. |

### EXAMPLE 2

An aliquot of 60 ml of 6% chlorine bleach poured onto a 4 ft×4 ft sample planar floor surface followed by a treatment of 100 grams of absorbent composition

| ABSORBENT COMPOSITION | |
|---|---|
| (% Weight basis) | OBSERVATIONS |
| 100% sodium acrylate crosslinked | Rapid liquid absorption. |
| homo-polymer with 10–15% | Strong Cl₂ odor detected. |
| extractables (200–800 μm range) | 122° F. max temp observed. |
| | Some residual sheen or slickness |
| | detected on floor after clean-up. |

### EXAMPLE 3

An aliquot of 60 ml of 6% chlorine bleach poured onto a 4 ft×4 ft sample planar floor surface followed by a treatment of 50 grams of absorbent composition.

| ADSORBENT COMPOSITION | |
|---|---|
| (% Weight basis) | OBSERVATIONS |
| 100% potassium acrylate | Rapid liquid absorption. |
| crosslinked homo-polymer | Mild Cl₂ odor detected. |
| | 128° F. max temp observed. |
| | Residual sheen or slickness detected |
| | on floor after clean-up. |

### EXAMPLE 4

An aliquot of 60 ml of 6% chlorine bleach poured onto a 4 ft×4 ft sample planar floor surface followed by a treatment of 50 grams of absorbent composition

| ADSORBENT COMPOSITION | |
|---|---|
| (% Weight basis) | OBSERVATIONS |
| 100% sodium acrylate crosslinked | Good liquid absorption. |
| homo-polymer with <10% | Some Cl₂ odor detected. |
| extractables (1500 μm avg with | 130° F. max temp observed. |
| 800–2500 μm range) | No sheen visible |
| | Good sweep up of swollen polymer |
| | particles. |

### EXAMPLE 5

An aliquot of 60 ml of 6% chlorine bleach poured onto a 4 ft×4 ft sample planar floor surface followed by a treatment of 100 grams absorbent composition

| ABSORBENT COMPOSITION | |
|---|---|
| (% Weight basis) | OBSERVATIONS |
| 100% zeolite | Rapid liquid absorption. |
| | Strong Cl₂ odor detected. |
| | 171° F. max temp observed. |
| | No residual sheen or slickness detected |
| | on floor after clean-up. |

### EXAMPLE 6

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 100% active carbon | Rapid liquid absorption. |
| | Minimal Cl₂ odor detected. |
| | 168° F. max temp observed. |
| | No residual sheen or slickness detected |
| | on floor after clean-up. |

### EXAMPLE 7

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 100% perlite | Poor liquid absorption. |
| | Strong Cl₂ odor detected. |
| | 84° F. max temp observed. |
| | No residual sheen or slickness detected |
| | on floor after clean-up. |

### EXAMPLE 8

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 100% sodium bentonite | Poor liquid absorption. |
| | Minimal Cl₂ odor detected. |
| | 84° F. max temp observed. |
| | No residual sheen or slickness detected |
| | on floor after clean-up. |

### EXAMPLE 9

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 100% silicon dioxide | Poor liquid absorption. |
| | Strong Cl₂ odor detected. |
| | 84° F. max temp observed. |
| | No residual sheen or slickness detected |
| | on floor after clean-up. |

### EXAMPLE 10

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 100% vermiculite | Poor liquid absorption. |
| | Strong Cl₂ odor detected. |
| | 82° F. max temp observed. |
| | No residual sheen or slickness detected |
| | on floor after clean-up. |

### EXAMPLE 11

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 100% acrylate-acrylamide co- | Rapid liquid absorption. |
| polymer | Strong Cl₂ odor detected and yellow gas |
| | cloud generated. |
| | 172° F. max temp observed. |
| | Residual sheen or slickness detected |
| | on floor after clean-up |

### EXAMPLE 12

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 100% sodium acrylate crosslinked | Good liquid absorption. |
| homo-polymer with <15% | Some Cl₂ odor detected. |
| extractables | 132° F. max temp observed. |
| | Residual sheen or slickness detected |
| | on floor after clean-up |

### EXAMPLE 13

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 100% sodium acrylate crosslinked | Good liquid absorption. |
| homo-polymer with <10% extractables | Some Cl₂ odor detected. |
| (1500 μm avg with 800–2500 μm range) | 132° F. max temp observed. |
| | No sheen visible |

### EXAMPLE 14

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 35% sodium acrylate crosslinked homo- | Rapid liquid absorption. |
| polymer with 10–15% extractables | Minimal Cl₂ odor detected. |
| 65% dried corn cob particles | 144° F. max temp observed. |
| (200–800 μm range) | Residual sheen or slickness |
| | detected on floor after clean-up |

### EXAMPLE 15

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 50% acrylate-acrylamide co-polymer | Rapid liquid absorption. |
| 50% vermiculite | Improved vapor suppression |
| | 145° F. max temp observed. |
| | Residual sheen or slickness |
| | detected on floor after clean-up |

### EXAMPLE 16

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 25% acrylate-acrylamide co-polymer | Slower liquid absorption. |
| 75% vermiculite | Less Cl₂ odor detected. |
| | 134° F. max temp observed. |
| | Residual sheen or slickness |
| | detected on floor after clean-up |

### EXAMPLE 17

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 50% sodium acrylate crosslinked homo- | Good liquid absorption. |
| polymer with <10% extractables | Little Cl₂ odor detected. |
| (1500 μm avg with 800–2500 μm range) | 89° F. max temp observed. |
| 50% vermiculite | No sheen visible |

### EXAMPLE 18

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 25% sodium acrylate crosslinked | Good liquid absorption. |
| homo-polymer with <10% extractables | Rapid vapor suppression. Little |
| (1500 μm avg with 800–2500 μm range) | Cl₂ odor detected. |
| 75% vermiculite | 82° F. max temp observed. |
| | No sheen visible |

### EXAMPLE 19

An aliquot of 60 ml of mouthwash poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 25% sodium acrylate crosslinked homo- | Good liquid absorption. |
| polymer with <10% extractables | No sheen visible. |
| (1500 μm avg with 800–2500 μm range) | Absorbed materials sweeps up |
| 75% vermiculite | easily. |

### EXAMPLE 20

An aliquot of 60 ml of liquid laundry detergent poured onto a 4 ft×4 ft sample planar floor surface followed by a treatment of 50 grams of absorbent composition then 10-20 mls tap water followed by a second 25 grams absorbent composition.

| ABSORBENT COMPOSITION | |
|---|---|
| (% Weight basis) | OBSERVATIONS |
| 25% sodium acrylate crosslinked | Fair absorption on initial application. |
| homo-polymer with <10% | Swept up in initial application. |
| extractables | Applied 20 mls tap water to spill area |
| (1500 μm avg with 800–2500 | then 25 grams fresh absorbent. |
| μm range) | Absorbed material swept up easily. No |
| 75% vermiculite | floor sheen visible. |

### EXAMPLE 21

An aliquot of 60 ml of 10W30 motor oil poured onto a 4 ft×4 ft sample planar floor surface followed by a treatment of 50 grams of absorbent composition then 10-20 mls tap water followed by a second 50 grams absorbent composition.

| ABSORBENT COMPOSITION | |
|---|---|
| (% Weight basis) | OBSERVATIONS |
| 25% sodium acrylate crosslinked | Fair absorption on initial application. |
| homo-polymer with <10% | Swept up in initial application. |
| extractables (1500 μm avg with | Applied 20 mls tap water to spill |
| 800–2500 μm range) | area then 50 grams fresh absorbent. |
| 75% vermiculite | Absorbed material swept up easily. |
| | No floor sheen visible. |

### EXAMPLE 22

An aliquot of 60 ml of milk poured onto a 4 ft×4 ft sample planar floor surface followed by a treatment of 75 grams of absorbent composition.

| ABSORBENT COMPOSITION | |
|---|---|
| (% Weight basis) | OBSERVATIONS |
| 25% sodium acrylate crosslinked | Excellent rapid absorption. |
| homo-polymer with <10% | Granular absorbent swept |
| extractables (1500 μm avg with | up easily and no visible floor |
| 800–2500 μm range) | sheen/slick spot. |
| 75% vermiculite | |

### EXAMPLE 23

An aliquot of 60 ml of household ammonia poured onto a 4 ft×4 ft sample planar floor surface followed by a treatment of 100 grams of absorbent composition.

| ABSORBENT COMPOSITION | |
|---|---|
| (% Weight basis) | OBSERVATIONS |
| 25% sodium acrylate crosslinked | Rapid suppression of vapor |
| homo-polymer with <10% extractables | with rapid absorption and easy |
| (1500 μm avg with 800–2500 μm range) | clean-up. |
| 75% vermiculite | |

### EXAMPLE 24

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 40% sodium acrylate crosslinked | Rapid liquid absorption. |
| homo-polymer | Minimal Cl₂ odor detected. |
| (500 μm avg with 200–900 | 187° F. max temp observed. |
| μm range) | Residual sheen or slickness detected |
| 35% dried corn cob particles | on floor after clean-up. |
| (200–800 μm range) | Swollen polymer particles escape from |
| 25% vermiculite | brush & swipe. |
| (150–900 μm range) | |

### EXAMPLE 25

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 35% sodium acrylate crosslinked | Rapid liquid absorption. |
| homo-polymer with 10–15% | Minimal Cl₂ odor detected. |
| extractables | 133° F. max temp observed. |
| 40% active carbon | Residual sheen or slickness detected |
| 25% vermiculite | on floor after clean-up |

### EXAMPLE 26

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 35% sodium acrylate crosslinked homo- | Rapid liquid absorption |
| polymer with <10% extractables | Minimal Cl₂ odor detected. |
| 40% active carbon | 134° F. max temp observed. |
| 25% vermiculite | No residual sheen or slickness |
| | detected on floor after clean-up. |

### EXAMPLE 27

An aliquot of 60 ml of 6% chlorine bleach poured onto a 4 ft×4 ft sample planar floor surface followed by a treatment of 100 grams absorbent composition.

| ADSORBENT COMPOSITION | |
|---|---|
| (% Weight basis) | OBSERVATIONS |
| 25% sodium acrylate crosslinked | Rapid liquid absorption. |
| homo-polymer with <10% extractables | Minimal Cl₂ odor detected. |
| (1500 μm avg with 800–2500 μm range) | 101° F. max temp observed. |
| 35% vermiculite | No residual sheen or slickness |
| (150–900 μm range) | detected on floor after clean-up. |
| 40% Corncob | Swollen polymer particles sweep |
| (200–800 μm range) | up easily with brush and |
| | wipe away. |

### EXAMPLE 28

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 35% sodium acrylate crosslinked homo- | Rapid liquid absorption. |
| polymer with <10% extractables | Minimal Cl₂ odor detected. |
| 30% dried corn cob particles | 122° F. max temp observed. |
| (200–800 μm range) | No residual sheen or slickness |
| 35% perlite | detected on floor after clean-up. |

### EXAMPLE 29

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 35% sodium acrylate crosslinked homo- | Rapid liquid absorption. |
| polymer with <10% extractables | Minimal Cl₂ odor detected. |
| 25% dried corn cob particles | 107° F. max temp observed. |
| (200–800 μm range) | No residual sheen or slickness |
| 40% vermiculite | detected on floor after clean-up. |
| | Very light/low bulk density. |

### EXAMPLE 30

An aliquot of 60 ml of 6% chlorine bleach poured onto a 4 ft×4 ft sample planar floor surface followed by a treatment of 50 grams of absorbent composition.

| ABSORBENT COMPOSITION | |
|---|---|
| (% Weight basis) | OBSERVATIONS |
| 25% sodium acrylate crosslinked | Rapid liquid absorption. |
| homo-polymer with <10% | Minimal Cl₂ odor detected. |
| extractables | 98° F. max temp observed. |
| 40% dried corn cob particles | No residual sheen or slickness detected |
| (200–800 μm range) | on floor after clean-up. |
| 35% perlite | Bulk density is workable for packaging. |

### EXAMPLE 31

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 15% sodium acrylate crosslinked homo- | Rapid liquid absorption. |
| polymer | Minimal Cl₂ odor detected. |
| (500 μm avg with 200–800 μm range) | 92° F. max temp observed. |
| 60% vermiculite | Residual sheen or slickness |
| (150–900 μm range) | detected on floor after clean-up |
| 25% dried corn cob particles | Swollen polymer particles |
| (200–800 μm range) | escape from brush & squeegee |

### EXAMPLE 32

A spilt of 60 ml of 6% chlorine bleach on a 4 ft×4 ft sample planar floor surface followed by an addition of 100 grams absorbent composition.

| ABSORBENT COMPOSITION | |
|---|---|
| (% Weight basis) | OBSERVATIONS |
| 25% sodium acrylate crosslinked homo- | Rapid liquid absorption. |
| polymer | Minimal Cl₂ odor detected. |
| (500 μm avg with 200–800 μm range) | 115° F. max temp observed. |
| 35% vermiculite | Residual sheen or slickness |
| (150–900 μm range) | detected on floor after clean-up |
| 40% dried corn cob particles | Swollen polymer particles |
| (200–800 μm range) | escape from brush & squeegee |

### EXAMPLE 33

An aliquot of 60 ml of 6% chlorine bleach poured onto a 4 ft×4 ft sample planar floor surface followed by a treatment of 100 grams absorbent composition

| ABSORBENT COMPOSITION | |
|---|---|
| (% Weight basis) | OBSERVATIONS |
| 25% sodium acrylate crosslinked homo- | Rapid liquid absorption. |
| polymer with <10% extractables | Minimal Cl₂ odor detected. |
| (500 μm avg with 200–800 μm range) | 115° F. max temp observed. |
| 35% Vermiculite | No residual sheen or slickness |
| (150–900 μm range) | detected on floor after clean-up. |
| 40% dried corn cob particles | Swollen polymer particles |
| (200–800 μm range) | escape from brush & squeegee |

### EXAMPLE 34

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 25% sodium acrylate crosslinked homo- | Rapid liquid absorption. |
| polymer with <10% extractables | Minimal Cl₂ odor detected. |
| (900 μm avg with 700–1400 μm range) | 115° F. max temp observed. |
| 35% vermiculite | No residual sheen or slickness |
| (150–900 μm range) | detected on floor after clean-up. |
| 40% dried corn cob particles | Some improvement in sweep-up |
| (200–800 μm range) | performance but some swollen |
| | polymer particles continue to |
| | escape from brush & swipe. |

### EXAMPLE 35

An aliquot of 60 ml of 6% chlorine bleach poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 25% sodium acrylate crosslinked homo- | Rapid liquid absorption. |
| polymer with <10% extractables | Minimal Cl₂ odor detected. |
| (1000 μm avg with 700–1400 μm range) | 101° F. max temp observed. |
| 35% vermiculite | No residual sheen or slickness |
| (150–900 μm range) | detected on floor after clean-up. |
| 40% dried corn cob particles | Some improvement in sweep-up |
| (200–800 μm range) | performance but some swollen |
| | polymer particles continue to |
| | escape from brush & swipe. |

### EXAMPLE 36

An aliquot of 50 ml of 6% sodium hypochlorite poured onto a 4 ft×4 ft sample planar floor surface followed by a treatment of 100 grams absorbent composition.

| ABSORBENT COMPOSITION | |
|---|---|
| (% Weight basis) | OBSERVATIONS |
| 25% sodium acrylate crosslinked homo- | Rapid liquid absorption. |
| polymer with <10% extractables | Minimal Cl₂ odor detected. |
| (1500 μm avg with 800–2500 μm range) | 88° F. max temp observed. |
| 35% vermiculite | No residual sheen or slickness |
| (150–900 μm range) | detected on floor after clean-up. |
| 40% dried corn cob particles | Ease in Sweeping up because |
| (200–800 μm range) | swollen particles easily swept up |
| | with no discernable slick or |
| | shine to floor. |

### EXAMPLE 37

An aliquot of 50 ml of 10/40 weight motor oil poured onto a 4 ft×4 ft sample planar floor surface followed by a treatment of 150 grams absorbent composition.

| ABSORBENT COMPOSITION | |
|---|---|
| (% Weight basis) | OBSERVATIONS |
| 25% sodium acrylate crosslinked | Rapid liquid absorption. |
| homo-polymer with <10% | No Cl₂ odor detected. |
| extractables | No change in temp observed. |
| (1500 μm avg with 800–2500 | Secondary residual sheen or |
| μm range) | slickness detected on floor after initial |
| 35% vermiculite | clean-up but secondary residual sheen |
| (150–900 μm range) | spraying water onto sheen and adding |
| 40% dried corn cob particles | additional 25 g of absorbent and water. |
| (200–800 μm range) | Ease in Sweeping up because |
| | swollen particles easily swept up with |
| | no discernable slick or shine to floor. |

### EXAMPLE 38

A 50 ml aliquot of 50 ml of a commercial cleaning product (containing linear alcohol ethoxylates and n-propoxypropanol) poured onto a 4 ft×4 ft planar floor surface followed by exposure to 150 grams absorbent composition.

| ABSORBENT | |
|---|---|
| COMPOSITION | |
| (% Weight basis) | OBSERVATIONS |
| 25% sodium acrylate | Rapid liquid absorption. |
| crosslinked homo-polymer | No Cl₂ odor detected. |
| with <10% extractables | No change in temp observed. |
| (1500 μm avg with 800–2500) | Secondary residual sheen or slickness |
| μm range | detected on floor after initial clean-up but |
| 35% vermiculite | secondary residual sheen spraying 25 ml |
| (150–900 μm range) | of water onto sheen and adding |
| 40% dried corn cob particles | additional 25 g of absorbent and water. |
| (200–800 μm range) | Ease in Sweeping up because swollen |
| | particles easily swept up with no |
| | discernable slick or shine to floor. |

### EXAMPLE 39

A 50 ml aliquot of 50 ml of a Aerosol lubricant (light fraction aliphatic petroleum distillate commercial product) poured onto a 4 ft×4 ft planar floor surface followed by exposure to 150 grams absorbent composition.

| ABSORBENT | |
|---|---|
| COMPOSITION | |
| (% Weight basis) | OBSERVATIONS |
| 25% sodium acrylate | Rapid liquid absorption. |
| crosslinked homo-polymer | No Cl₂ odor detected. |
| with <10% extractables | No change in temp observed. |
| (1500 μm avg with | Secondary residual sheen or slickness |
| 800–2500 μm range) | detected on floor after initial clean-up but |
| 35% vermiculite | secondary residual sheen spraying 10 ml |
| (150–900 μm range) | of water onto sheen and adding |
| 40% dried corn cob particles | additional 25 g of absorbent and water. |
| (200–800 μm range) | Ease in Sweeping up because swollen |
| | particles easily swept up with no |
| | discernable slick or shine to floor. |

### EXAMPLE 40

A 50 ml aliquot of 50 ml of a aerosol lubricant (light fraction aliphatic petroleum distillate commercial product) poured onto a 4 ft×4 ft planar floor surface followed by exposure to 100 grams absorbent composition.

| ABSORBENT COMPOSITION | |
|---|---|
| (% Weight basis) | OBSERVATIONS |
| 25% sodium acrylate crosslinked | Rapid liquid absorption. |
| homo-polymer with <10% | Rapid suppression of ammonia odor. |
| extractables (1500 μm avg | No change in temp observed. |
| with 600–2500 μm range) | Ease in Sweeping up because swollen |
| 35% vermiculite | particles easily swept up with no |
| (150–900 μm range) | discernable slick or shine to floor. |
| 40% dried corn cob particles | |
| (200–800 μm range) | |

As to the manner of usage and operation of the present invention, the same should be apparent from the above description. Accordingly, no further discussion relating to the manner of usage and operation will be provided.

While a preferred embodiment of the absorbent article has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An absorbent article comprising:
a super-absorbent polymer comprising about 20% to about 80% by weight of the absorbent article wherein
the polymer is composed of a lightly-crosslinked sodium polyacrylate having less than 15% by weight of extractables remaining in the lightly-crosslinked sodium polyacrylate, and
the extractables are defined as residues extracted from the lightly-crosslinked sodium polyacrylate using an extracting solvent; and
a heat-sink material composed of vermiculite comprising about 20% to about 80% by weight of the absorbent article.

2. The absorbent article of claim 1 further comprising a vapor suppressant.

3. The absorbent article of claim 2 wherein the vapor suppressant is corncob particles.

4-6. (canceled)

7. The absorbent article of claim 6 wherein the polyacrylate having less than 10% by weight of extractables.

8. (canceled)

9. The absorbent article of claim 1 wherein the extracting solvent is n-hexane.

10. The absorbent article of claim 1 wherein the absorbent article has an overall absorbent capacity of over 60 % by weight of the absorbent article.

11. The absorbent article of claim 1 wherein the absorbent article has an overall absorbent capacity of over 150 % by weight of the absorbent article.

12. The absorbent article of claim 1 wherein the super-absorbent polymer is present in an amount of about 25% to about 40% by weight of the absorbent article.

13. The absorbent article of claim 1, wherein the heat-sink material is present in an amount of about 50% to about 80% by weight of the absorbent article.

14. The absorbent article of claim 2, wherein the vapor suppressant is present in an amount of 20% to about 80% by weight of the absorbent article.

15. The absorbent article of claim 1 wherein the super-absorbent polymer having a majority of particles sized to be greater than 700 μm in diameter.

16. The absorbent article of claim 1 wherein the heat-sink material having a specific heat of at least 0.5 kJ/KgK.

17. The absorbent article of claim 1 wherein the heat-sink material comprises vermiculite having a specific heat of at least 0.8 kJ/kgK.

18. The absorbent article of claim 1 wherein a reaction temperature of no more than 150° F. is generated when equal amounts of the absorbent article and a spilled liquid are mixed together

19. The absorbent article of claim 1 wherein a reaction temperature of no more than 150° F. is generated when approximately equal amounts of the absorbent article and a spilled liquid are mixed together wherein the spilled liquid is a chlorine bleach product.

20. An absorbent article comprising:
a super-absorbent polymer in an amount of about 20% to about 80% by weight of the absorbent article, the super-absorbent polymer comprising a lightly-crosslinked sodium polyacrylate having less than 15% by weight of extractables in the super-absorbent polymer wherein the extractables comprise residues extracted from the super-absorbent polymer using an extracting solvent;
a heat-sink material in an amount of about 20% to about 80% by weight of the absorbent article, the heat-sink material having a specific heat of at least 0.5 kJ/KgK wherein the heat-sink material is vermiculite; and
a vapor suppressant in an amount of about 20% to about 80% by weight of the absorbent article wherein the vapor suppressant is corncob particles,
wherein a reaction temperature of no more than 150° F. is generated when approximately equal amounts of the absorbent article and a spilled liquid are mixed together.
